(19) United States
(12) Patent Application Publication
(10) Pub. No.: US 2010/0228327 A1
(43) Pub. Date: Sep. 9, 2010

(54) PORTABLE THERAPEUTIC INSTRUMENT FOR CORONARY HEART DISEASE

(76) Inventor: Zhiyong Cen, Guangzhou City, Guangdong Province (CN)

Correspondence Address: Guangzhou Scihead Patent Agent Co., LTD., Room 1508, Huihua Commercial Trading Buliding, No. 80, Shengleichung road, Guangzhou, 510070, omitted

(21) Appl. No.: 12/680,578

(22) PCT Filed: Sep. 30, 2007
(86) PCT No.: PCT/CN07/02876

§ 371 Date: Mar. 29, 2010

(51) Int. Cl. A61N 5/067

(52) U.S. Cl. 607/89

(57) ABSTRACT

A portable therapeutic instrument for coronary heart disease comprises a main body and a semiconductor laser disposed in the main body. The main body includes an upper housing and a lower housing connected with each other. Finger-fixing sheathes are disposed on the lower housing. The finger-fixing sheathes consist of three upright dividers. Near the bottom of the leading edge of each of the three upright dividers is centrally disposed a laser transmission protruding-hole. A laser light power control circuit, a time control circuit, a charge control circuit, and a display control circuit are disposed in the main body.

### BACKGROUND OF THE INVENTION

1. Technical Field

The present invention relates to a medical equipment, and more particularly, to a therapeutic instrument for coronary heart disease.

2. Description of Related Art

China Patent Publication No. CN2642343Y is a utility model patent that discloses a therapeutic instrument for coronary heart disease, comprising a photon glove and a main body apart from the photon glove, and, as a result, the photon glove and the main body have to be connected by a wire, thus bringing inconvenience to users.

### BRIEF SUMMARY OF THE INVENTION

In view of the drawbacks of the existing art, it is an objective of the present invention to provide a portable therapeutic instrument for coronary heart disease such that the portable therapeutic instrument for coronary heart disease is easy to assemble, easy to operate, and convenient to carry.

To achieve the above objective, the present invention provides a portable therapeutic instrument for coronary heart disease, comprising a main body and a semiconductor laser disposed in the main body. The main body comprises an upper housing and a lower housing connected with each other. Finger-fixing sheathes are disposed on the lower housing. The finger-fixing sheathes consist of three upright dividers. Near the bottom of the leading edge of each of the three upright dividers is centrally disposed a laser transmission protruding-hole. A laser light power control circuit, a time control circuit, a charge control circuit, and a display control circuit are disposed in the main body.

The middle one of the three upright dividers is a fixed upright divider, and the two side ones are movable upright dividers.

The movable upright dividers are formed by plastic injection or made by elastic material.

The upright dividers are spaced apart from each other by a relatively large space. The middle upright divider is covered with a soft plastic case of a shape matching the middle upright divider.

The middle upright divider is slightly behind the side upright dividers.

The laser transmission protruding-holes are hollow and receive optical fibers. The optical fibers are connected to the semiconductor lasers disposed in the main body.

The laser transmission protruding-holes are of a diameter that ranges between 1.1 mm and 1.2 mm. The optical fibers are of a diameter of 1.0 mm.

The semiconductor laser is a 650 nm or 670 nm wavelength semiconductor laser.

Compared with the existing art, the present invention provides a portable therapeutic instrument for coronary heart disease, comprising a main body having an upper housing and a lower housing. The upper housing has a panel provided with a control component and a display screen. The lower housing has finger-fixing sheathes and laser transmission protruding-holes. The main body is provided therein with three said semiconductor lasers, a power source, a plurality of circuit boards (including a charge circuit, a laser power control circuit, a time control circuit, a display circuit, and a control circuit), an operation panel having an operation button and a display screen, and a charge jack. Hence, the portable therapeutic instrument for coronary heart disease of the present invention is easy to operate and convenient to carry.

### BRIEF DESCRIPTION OF THE SEVERAL VIEWS OF THE DRAWINGS

The present invention is further illustrated with a specific embodiment in conjunction with the accompanying drawings, in which:

FIG. 1 is a perspective view of a lower housing of a main body according to the present invention;

FIG. 2 is a schematic view of an upper housing of the main body according to the present invention;

FIG. 3 is a schematic view of the upper housing according to the present invention;

FIG. 4 is a perspective view of the lower housing according to the present invention;

FIG. 5 is a schematic view of a laser light power control circuit board according to the present invention;

FIG. 6 is a schematic view of a time, charge, display control circuit board according to the present invention;

FIG. 7 is a schematic view of a chip control circuit diagram according to the present invention;

FIG. 8 is a laser output driving circuit diagram according to the present invention.

### DETAILED DESCRIPTION OF THE INVENTION

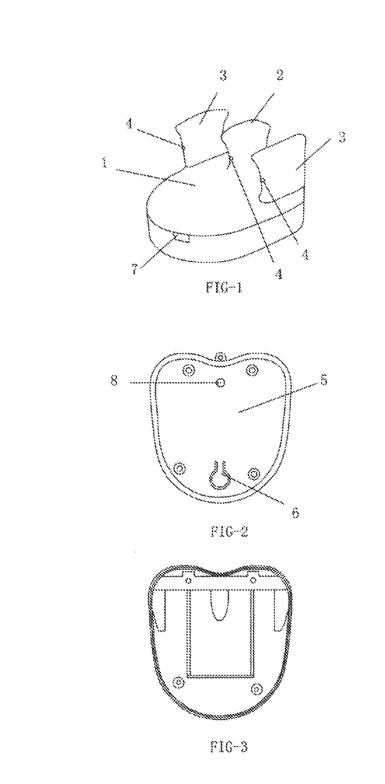

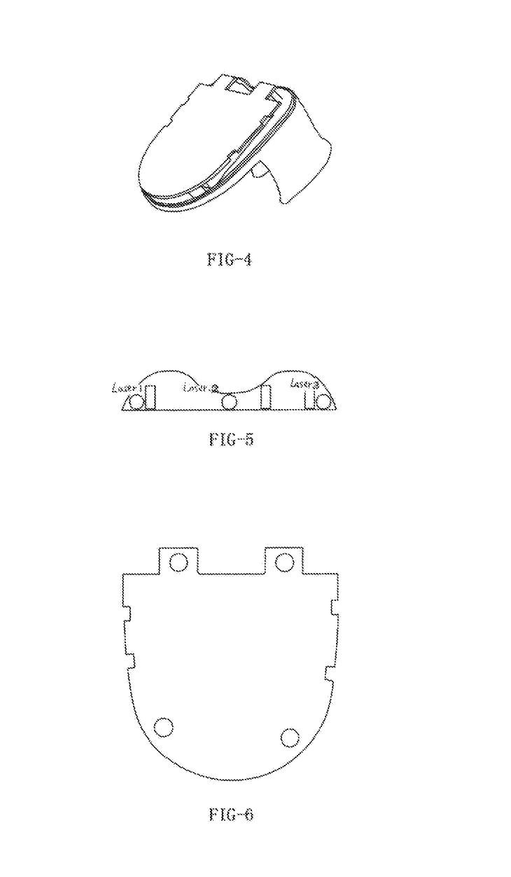

Referring to FIG. 1 through FIG. 4, a portable therapeutic instrument for coronary heart disease of the present invention comprises a main body 1, finger-fixing sheathes 2, 3, laser transmission protruding-holes 4, a panel 5, a switch button 6, a charge jack 7, and an operation indicator 8. The main body 1 comprises an upper housing and a lower housing connected and fixed to each other by screws or any other mechanical means. The finger-fixing sheathes 2, 3 disposed on the lower housing consist of three upright dividers, wherein the middle upright divider 2 is fixed in position while the two side upright dividers 3 are movable. The movable upright dividers 3 are formed by performing injection molding on plastics or soft plastics and configured to move sideways to a certain extent so as to suit different sizes of fingers of different persons. Upon completion of adjustment, fixation is achieved by screwing or engagement. In another embodiment, during the die fabricating stage, the inner space between the finger-fixing sheathes 2, 3 is expanded so as to suit users with thick fingers, and the middle upright divider 2 is covered with a soft plastic case corresponding in shape to the middle upright divider 2 so as to suit users with thin fingers. Near the bottom of the leading edge of each of the three upright dividers is centrally disposed a laser transmission protruding-hole 4. Once a user's fingers penetrate the finger-fixing sheathes 2, 3 and hold the main body 1 gently, the laser transmission protruding-holes 4 will come into contact with acupuncture points beneath the skin connected between the index finger, the middle finger, the ring finger, and the little finger. The laser transmission protruding-holes 4 not only stimulate the acupuncture points but also press against the skin to thereby prevent leakage of laser emitted from at least one semiconductor laser. In the present embodiment, there are three semiconductor lasers. The interphalangeal acupuncture points are the important ones of the acupuncture points associated with Heart Meridian and Pericardium Meridian according to the theory of meridian pathways taught in traditional Chinese medicine. As verified in clinical tests, when the acupuncture points are irradiated by 650 mm laser, laser penetrates subcutaneous tissue to reach capillaries and arterioles and thus, due to a photoelectrical field and light energy conversion, enhances deformation of red blood cells, increases oxygen-carrying capacity of red blood cells, improves microcirculation, and ultimately promote the supply of blood and oxygen to the heart and the brain.

The laser transmission protruding-holes 4 protrude by a distance of 1.0˜1.5 mm approximately. A human anatomical feature is that the skin between the index finger and the middle finger and the skin between the middle finger and the ring finger are farther from the wrist than the skin between the ring finger and the little finger. Hence, the middle upright divider 2 has to be slightly behind the side upright dividers 3 in order to suit the aforesaid anatomical feature. The laser transmission protruding-holes 4 are hollow and are of a diameter that ranges between 1.1 mm and 1.2 mm. The laser transmission protruding-holes 4 receive optical fibers of a diameter of 1.0 mm approximately. The optical fibers penetrate inner holes of the upright dividers to connect with the semiconductor lasers disposed in the main body 1. The semiconductor laser in operation emits laser for irradiating the optical fibers, and the emitted laser is transmitted to the laser transmission protruding-holes 4 by the optical fibers so as to irradiate the acupuncture beneath the skin between the fingers.

A rechargeable battery (not shown) is disposed in the main body 1 to supply electric power to the semiconductor lasers and various control circuits. The charge jack 7 is disposed on one side of the main body 1 such that the charge jack 7 is opposite to the finger-fixing sheathes 2, 3. The charge jack 7 is configured for use with a dedicated charger for charging the rechargeable battery.

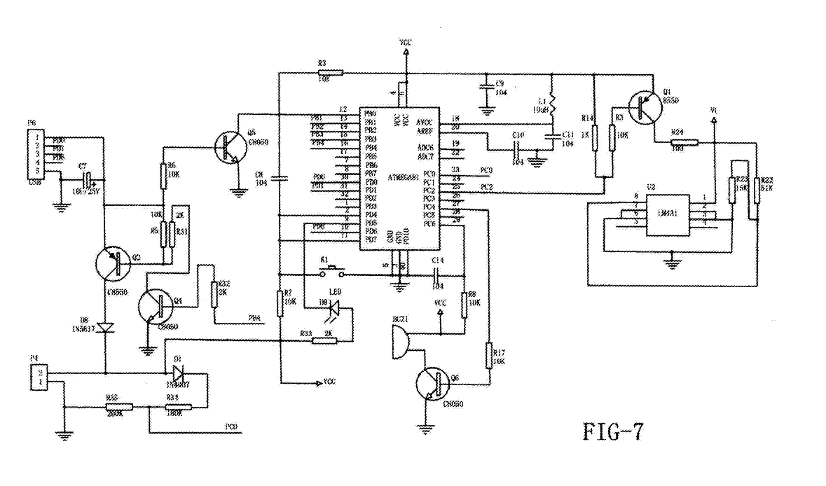

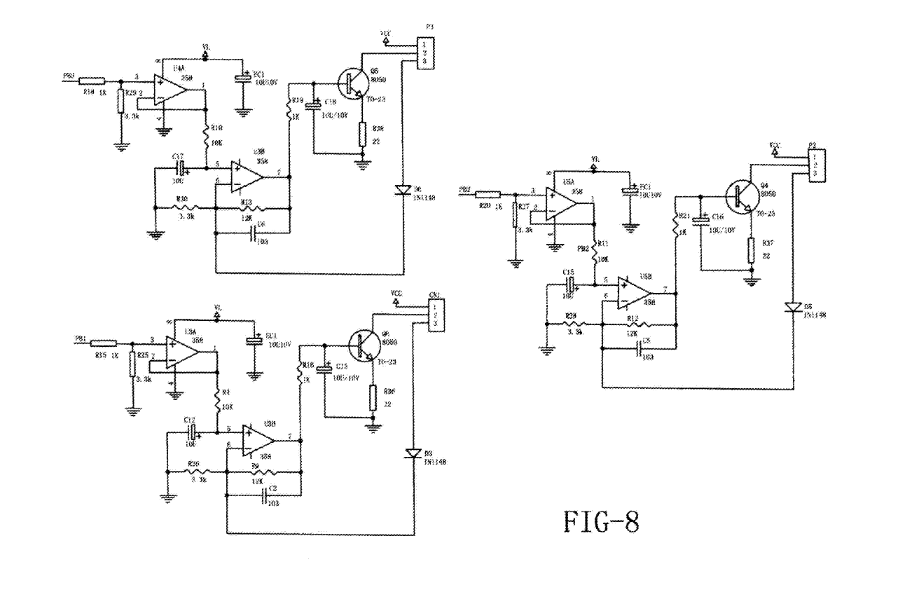

Referring to FIG. 5 through FIG. 8, a control circuit board (not shown) is disposed at the bottom of the upper housing of the main body. Disposed at the upper portion of the control circuit board are an oblong laser light power control circuit board and a semicircular time, charge, and display control circuit board, providing a time control circuit U6, a charge control circuit U6+P4, a display control circuit U6+D9, and laser light power control circuits P1, P2, P3.

In an embodiment of the present invention, the main body 1 is held by a user's palm, and the finger-fixing sheathes 2, 3 which have been well-adjusted are penetrated by the middle finger and the ring finger. After feeling comfortable with the finger-fixing sheathes 2, 3, the user's fingers press the main body 1 gently such that the laser transmission protruding-holes 4 align with the acupuncture points beneath the skin between the index finger, middle finger, ring finger, and little finger, respectively. Then, the user presses the switch button 6 on the panel 5 for three seconds to trigger the semiconductor lasers in the main body 1 to emit laser. The emitted lasers are transmitted to the laser transmission protruding-holes 4 by the optical fibers to thereby irradiate the acupuncture beneath the skin.

The semiconductor laser of the present invention has a wavelength that ranges between 500 nm and 760 nm. The portable therapeutic instrument for coronary heart disease of the present invention selectively uses a 650 nm or 670 nm semiconductor laser.

What is claimed is:

1. A portable therapeutic instrument for coronary heart disease, comprising a main body and at least one semiconductor laser disposed in the main body, and characterized in:
the main body comprising an upper housing and a lower housing connected with each other, the lower housing having finger-fixing sheathes comprising three upright dividers, wherein near a bottom of a leading edge of each of three said upright dividers is centrally disposed a laser transmission protruding-hole, and the main body being provided therein with a laser light power control circuit, a time control circuit, a charge control circuit, and a display control circuit.

2. The portable therapeutic instrument of claim 1, wherein in the three upright dividers, the middle upright divider is fixed and the two side upright dividers are movable, the movable upright dividers being made of plastics or elastic material by plastic injection.

3. The portable therapeutic instrument of claim 1, wherein the upright dividers are spaced apart from each other by a relatively large space, with the middle upright divider being covered with a soft plastic case of a shape matching the middle upright divider.

4. The portable therapeutic instrument of claim 1, wherein the middle upright divider is slightly behind the side upright dividers.

5. The portable therapeutic instrument of claim 1, wherein the laser transmission protruding-holes are hollow and receive optical fibers connected to the semiconductor laser disposed in the main body, the laser transmission protruding-holes being of a diameter ranging between 1.1 mm and 1.2 mm, and the optical fibers being of a diameter of 1.0 mm.

6. The portable therapeutic instrument of claim 1, wherein the semiconductor laser is a 650 nm or 670 nm wavelength semiconductor laser.
